(19) United States
(12) Patent Application Publication
(10) Pub. No.: US 2019/0184574 A1
(43) Pub. Date: Jun. 20, 2019

(54) SYSTEMS AND METHODS FOR AUTOMATED REHABILITATION

(71) Applicants: Chao-Wei Chen, Kaohsiung City (TW); Chih-Jui Ray Chen, Yilan County (TW); Co Yih Siow, Kaohsiung City (TW)

(72) Inventors: Chao-Wei Chen, Kaohsiung City (TW); Chih-Jui Ray Chen, Yilan County (TW); Co Yih Siow, Kaohsiung City (TW)

(21) Appl. No.: 16/130,276

(22) Filed: Sep. 13, 2018

Related U.S. Application Data
(60) Provisional application No. 62/557,758, filed on Sep. 13, 2017.
(60) Provisional application No. 62/655,237, filed on Apr. 10, 2018.

(51) Int. Cl. B25J 11/00; A61H 1/02; A61G 15/00; A61G 15/02; G16H 20/30; B25J 9/16

(52) U.S. Cl. CPC B25J 11/009; A61H 2201/5048; A61G 15/007; A61G 15/02; G16H 20/30; B25J 9/1697; B25J 9/1661; B25J 9/1669; A61H 2201/1659; A61H 2201/501; A61H 2201/5092; A61H 2230/605; A61H 2201/5061; A61H 2201/5043; A61H 1/02

(57) ABSTRACT

The present invention provides a system and methods for automated rehabilitation. The system and methods could provide the automated coordination training and assessment.

### CROSS REFERENCE TO RELATED APPLICATIONS

This application claims the benefit of U.S. Provisional Patent Application Nos. 62/557,758, filed on Sep. 13, 2017 and 62/655,237, filed on Apr. 10, 2018, the entire disclosures of which are hereby incorporated by reference.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The present invention is related to the field of rehabilitation devices. More particularly, the invention relates to a rehabilitation system and methods that can provide automated rehabilitation services.

### BACKGROUND

Appropriate rehabilitation therapy is essential to promote recovery of many diseases, e.g. stroke, brain injury, Parkinson's disease. Both active and passive rehabilitation of musculoskeletal system can help maintain neurological and musculoskeletal function in patients. It also improves the quality of life. When done appropriately, rehabilitation therapy can prevent limbs spasticity, promote recovery of muscle strength, and promote recovery of limbs coordination. Commonly performed rehabilitation therapy includes the passive range of motion exercises, muscle strength training, and coordination training. However, rehabilitation service is a labor intensive process. Moreover, multiple factors can often limit the accessibility of conventional rehabilitation process.

A rehabilitation device with outward appearance and limbs structure mimicking part of the body of a human would have numerous benefits, such as greater flexibility (in particular when comparing to rehabilitation devices based on exoskeletal apparatus) and greater ease of use. The similarity of the device's outward appearance to the true human would also induce a positive emotional feeling in certain patients, and promote the patients' willingness to use the device. The device could work for a long time and could be quite precise in its assessments.

### SUMMARY OF THE DISCLOSURE

In some embodiments, a method of performing automated rehabilitation is provided. The method comprises receiving data from one or more optical sensor; determining a position of a part of the human body based on the data acquired by the optical sensor; and controlling one or more robotic arms to mobilize a part of the human body based on the position of a part of the human body as determined by the processor.

In some embodiments, controlling one or more robotic arms comprises controlling the one or more robotic arms to perform a passive range of motion exercises by mobilizing a part of the human body in a reciprocal motion. Controlling one or more robotic arms can comprise controlling the one or more robotic arms to perform muscle strength training by applying resistance against exertion. In some embodiments, controlling one or more robotic arms comprises controlling the one or more robotic arms and one or more optical sensor to perform coordination assessment by analyzing limb movement in a certain path while interacting with one or more robotic arms. The method can comprise selecting a training routine based on the coordination assessment. In some embodiments, the method comprises adjusting a training routine based on the coordination assessment. Controlling one or more robotic arms can comprise controlling the one or more robotic arms to perform coordination training by training a human to move limbs in a certain path by interacting with one or more robotic arms. In some embodiments, the method comprises adjusting the coordination training based on a coordination assessment. Controlling the one or more robotic arms can comprise controlling the one or more robotic arms to assess muscle strength by measuring the force of exertion against resistance. In some embodiments, the method comprises selecting a training routine based on the coordination assessment. The method can comprise adjusting a training routine based on the coordination assessment. In some embodiments, the method comprises generating a rehabilitation report based on data obtained during rehabilitation. The method can comprise determining a position of a part of the human body using machine learning. In some embodiments, the method comprises adjusting therapy administered by the system. The method can comprise determining latency time, velocity, acceleration, accuracy, smoothness, and/or submovement based on the patient's limb movement. In some embodiments, the method comprises changing a velocity of the robotic arm's movement, complexity of the robotic arm's movement pattern, and/or the latency time of the robotic arm's movement based on analysis of limb movement.

In some embodiments, a device for automated rehabilitation is provided. The device comprises at least one robotic arm comprising a force sensor, at least one link, at least one effector, and at least one joint, with at least one degree of freedom for each joint; an optical sensor; a processor; and memory coupled to the processor and storing instructions executable by the processor configured to cause the processor to: receive data from the optical sensor; determine a position of a part of the human body based on the data acquired by the optical sensor; and control one or more robotic arms to mobilize a part of the human body based on a position of a part of the human body as determined by the processor.

In some embodiments, the device comprises instructions executable by the processor configured to cause the processor to control the one or more robotic arms to perform an automated passive range of motion exercises by mobilizing a part of the human body in a reciprocal motion. The device can comprise instructions executable by the processor configured to cause the processor to select a passive range of motion exercise based on data acquired by the optical sensor. In some embodiments, the device comprises instructions executable by the processor configured to cause the processor to adjust a passive range of motion exercise based on data acquired by the optical sensor. The device can comprise additional instructions executable by the processor configured to cause the processor to perform muscle strength training by applying resistance against exertion. In some embodiments, the device comprises additional instructions executable by the processor configured to cause the processor to control the one or more robotic arms to perform coordination assessment by analyzing limb movement in a certain path interacting with one or more robotic arms. The device can comprise instructions executable by the processor configured to cause the processor to adjust an exercise based on the coordination assessment. In some embodiments, the device comprises instructions executable by the processor to cause the processor to control one or more robotic arms to perform coordination training by training a user to move limbs in a certain path by interacting with one or more robotic arms. The device can comprise instructions executable by the processor to cause the processor to adjust the coordination training based on data acquired by the optical sensor. In some embodiments, additional instructions executable by the processor cause the processor to assess muscle strength by measuring the force of exertion against resistance. The device can comprise instructions executable by the processor configured to cause the processor to generate a rehabilitation report from data received by the device. In some embodiments, the device comprises instructions executable by the processor configured to cause the processor to determine a position of a part of the human body using machine learning. The device can comprise at least one height adjustable platform. In some embodiments, the device comprises at least one display unit. The device can comprise at least one speaker. In some embodiments, the device comprises at least one mobile chair. The mobile chair can be electronically controllable. In some embodiments, the mobile chair is height adjustable, rotatable and can be reclined. The device can comprise at least one detachable marker that can detect the position a part of the human body. In some embodiments, the device comprises at least one connection to a network. The device can comprise instructions executable by the processor configured to cause the processor to determine latency time, velocity, acceleration, accuracy, smoothness, and/or submovement based on the patient's limb movement. In some embodiments, the device comprises instructions executable by the processor configured to cause the processor to change a velocity of the robotic arm's movement, complexity of the robotic arm's movement pattern, and/or the latency time of the robotic arm's movement based on analysis of limb movement. The optical sensor can comprise a depth sensor.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1a-c illustrates exemplary designs of the automated rehabilitation system.

FIG. 1d illustrates an exemplary design of the robotic arm.

FIG. 1e illustrates an exemplary design of an automated rehabilitation system.

FIG. 2 is a flowchart of a method for determination of anatomical structure in accordance with an exemplary embodiment.

FIG. 3 illustrates one embodiment of an automated rehabilitation service platform.

FIG. 4 is a flowchart of a method for determination of the range of motion in accordance with an exemplary embodiment.

FIG. 5 depicts one example of determining the range of motion of the right shoulder joint abduction of a patient.

FIG. 6 is a flowchart of a method for functional assessment in accordance with an exemplary embodiment.

FIG. 7 depicts one example of determining the strength of the right shoulder joint abduction of a patient.

FIG. 8 is a flowchart of a method for performing the automated passive range of motion exercises in accordance with an exemplary embodiment.

FIG. 9 depicts one example of performing the automated passive range of motion exercises for the abduction-adduction movement of the right shoulder joint.

FIG. 10 is a flowchart of a method for performing automated strength training in accordance with an exemplary embodiment.

FIG. 11 depicts one example of performing automated strength training for the flexion movement of the left elbow joint.

FIG. 12 is a flowchart of a method for performing coordination assessment in accordance with an exemplary embodiment.

FIG. 13 is a flowchart of a method for performing coordination training in accordance with an exemplary embodiment.

FIG. 14 depicts an exemplary computer system that, in some embodiments, can serve as the control system for the automated rehabilitation system.

FIG. 15 illustrates one embodiment of an automated rehabilitation service platform.

FIG. 16 is a flowchart of a method for performing coordination assessment in accordance with an exemplary embodiment.

FIG. 17 depicts one example of performing coordination assessment of the left upper limb of a patient.

FIG. 18 is a flowchart of a method for performing coordination training in accordance with an exemplary embodiment.

FIG. 19 depicts one example of performing coordination training of the left upper limb of a patient.

### DETAILED DESCRIPTION

Disclosed herein are embodiments of systems and methods for performing physical therapy using an automated system. Physical therapy often involves a therapist providing a movement or exercise for the patient to perform. The therapist often demonstrates the movement or exercise themselves, using their limbs to perform or direct the required movement. These motions and exercises are repeated over and over to train the muscles. Recent studies have shown that as much as 6-7 hours of physical therapy a day can be useful in regaining function. A human therapist is not be able to maintain precise consistency in their movements, especially when performing exercises for this length of time. An automated system provides the benefits of consistency and persistence. An automated system also provide the benefit of accurate perception of the patient. A human therapist is not able to discern the particularities of a patient's performance as accurately as is a machine. Thus, the automated system is able to better assess the patient's current level of ability. An automated system is also better able to track the patient's progress over time by comparing performance at different times.

The automated system described herein can comprise one or more sensors and a robotic system, such as robotic arms. The robotic arms can be used to indicate a desired motion and direct a patient through the exercise. The sensors can be used to detect patient performance. Analysis of the detected performance allows for shaping or adjusting of the therapy depending on perceived performance.

In some embodiments, the sensor used is an optical sensor. The optical sensor can be a two dimensional or a three dimensional sensor. For example, in some embodiments, the optical sensor comprises a two dimensional optical sensor comprising a depth sensor. Three dimensional perception allows the system to more accurately perceive the patient's motion. In some embodiments, the optical sensor comprises a two-dimensional camera similar to a webcam with an added sensor (e.g., like Microsoft Kinect, Intel® Real Sense™) comprising an infrared projector (depth sensor). In some embodiments, the optical sensor can produce an image (e.g., an RGB image) and depth information (e.g., a depth map with distance information). The frequency of image capture can be about 0-30 fps.

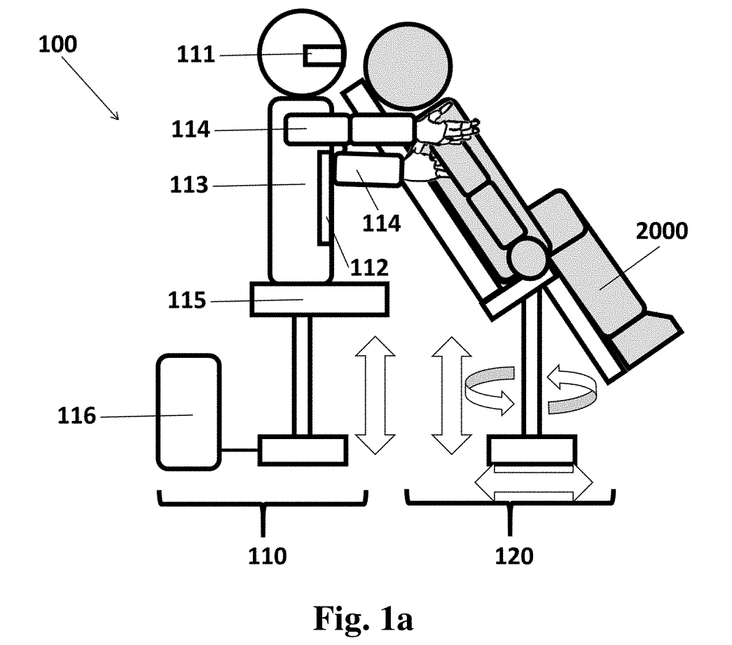

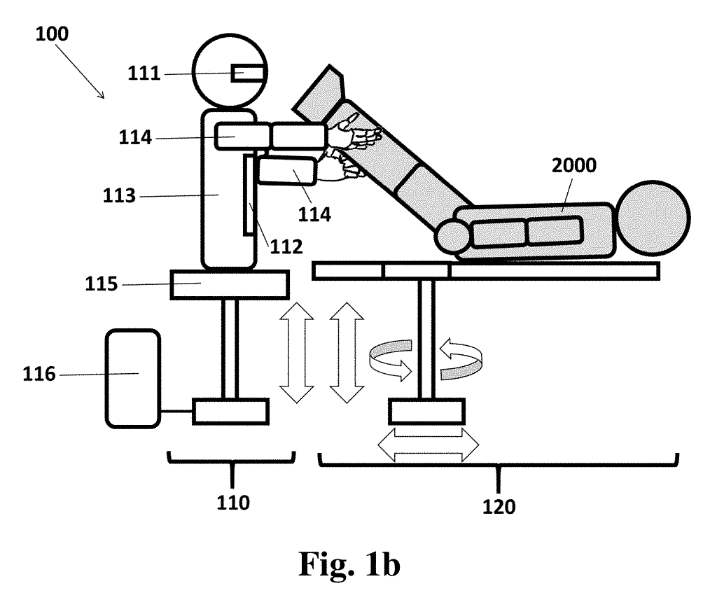

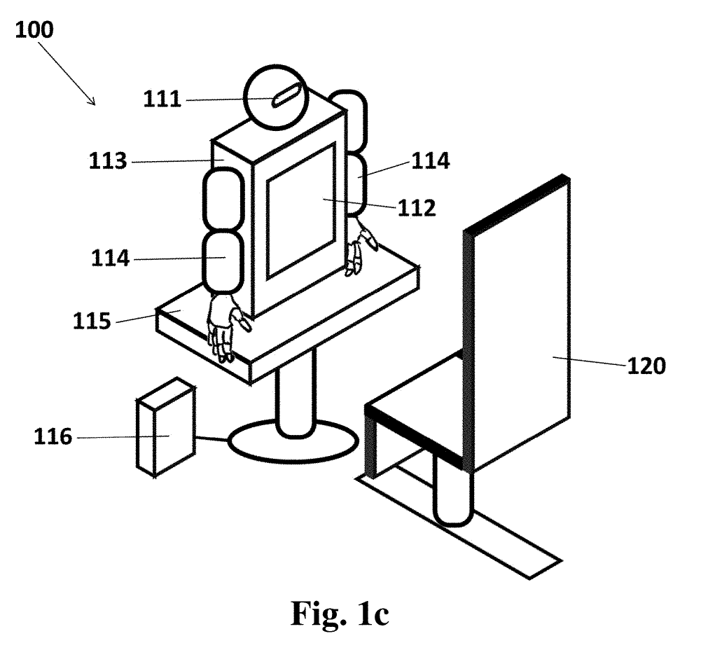

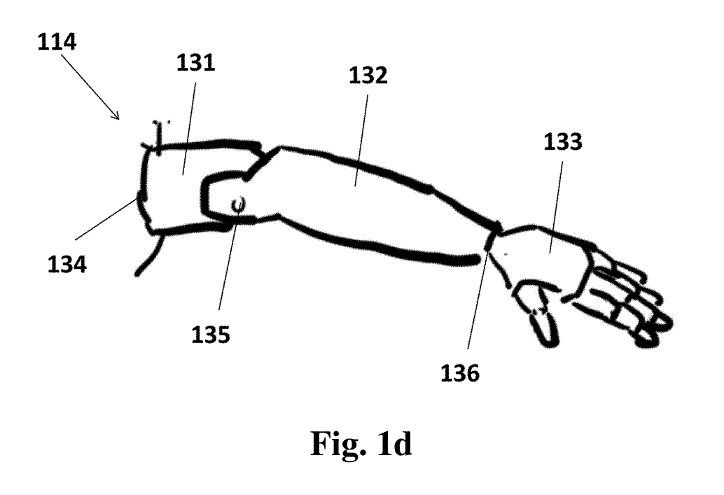

FIG. 1a illustrates an exemplary design of the automated rehabilitation system 100 with a patient 2000. In the embodiment shown, the system 100 comprises a robotic system 110 and a mobile chair system 120. In some embodiments, the system 100 does not comprise the mobile chair system. The system 100, in some embodiments, can be a device for automated rehabilitation.

The robotic system 110, in some embodiments, includes at least one optical sensor 111, at least one display unit 112, at least one speaker 113, at least one robotics arm 114, a platform 115, and a control system 116. In some embodiments, as shown in FIGS. 1a-1e, the system 100 comprises two robotic arms.

The optical sensor 111, in some embodiments, may incorporate a depth sensor, as described above.

The display unit 112, in some embodiments, can be a touch sensitive screen that can receive touch input from a user. Other configurations are also possible. For example, in some embodiments, the screen is not a touch sensitive screen. In some embodiments, the user can interface with the system using control buttons, a separate keyboard or keypad, or through a smartphone, tablet or computer.

The robotic arm 114, in some embodiments, can have at least one position sensors, and can have at least one force sensors. In some embodiments, the position sensor is used instead of or in conjunction with the optical sensor to determine the position of the patient's limbs. The force sensor can include a force sensor and/or a force-torque sensor. The force sensor can be placed in the effector, the joints, and/or even on the surface of the robotic arms. The force sensor can be used to detect muscle strength of the patients and/or to detect join range of motion of the patient. The robotic arm can comprise a seven DOF robotic arm. Other configurations are also possible (e.g., 3, 4, 5, 6, 8, 9, 10, or more DOF). In some embodiments, the robotic arm can have at least one motor for movement.

The platform 115, in some embodiments, can be height-adjustable, allowing better access to the patient's limbs. The platform can be automatically adjustable or manually adjustable. In some embodiments, the platform is not height-adjustable.

The control system 116, in some embodiments, can connect to a network and allow telerehabilitation. In some embodiment, the control system 116 can be operated remotely via the network connection.

The mobile chair system 120, in some embodiments, can be mobile, reclinable, height-adjustable, rotatable, and/or can be operated electronically. In the embodiment shown in FIG. 1a, the mobile chair system 120 is in the sitting mode, and the patient 2000 is sitting. The mobile chair system 120 may be driven by motors. In some embodiments, one or more detachable markers may be placed on one or more limbs and/or trunk of the patient 2000 to assist the system 100 in determining the position of the joint(s). In some embodiments, the system 100 is also capable of determining the position of the joint(s) without the use of the marker(s) (e.g., using only the optical sensor).

The control system 116, in some embodiments, can receive data from the optical sensors, the position sensors, the force sensors and the touch sensitive screen. In some embodiments, the control system can transmit data to the display unit 112 to display images, and transmit data to the speaker 113 to emit sounds. In some embodiments, the control system can transmit data to the robotic arm(s) 114, the platform 115, and the mobile chair system 120 to drive its movement.

FIG. 1b further illustrates an exemplary design of the automated rehabilitation system 100 with a patient 2000. In the embodiment shown 100, the mobile chair system 120 is in recline mode, and the patient 2000 is in a supine position. The system, in some embodiment, is able to provide rehabilitation services for both upper and lower limbs.

FIG. 1c further illustrates an exemplary design of the automated rehabilitation system 100 in another view.

FIG. 1e illustrates an exemplary design of the automated rehabilitation system 100 engaging with a patient 2000 such that the automated rehabilitation system is facing the patient 2000 and guiding the patient 2000 through an arm exercise. The system 100 of FIG. 1e differs from that shown in FIGS. 1a-1c as the system 100 of FIG. 1e does not include a platform 115. It will be appreciated that any of the embodiments described herein can be used with or without the platform.

FIG. 1d illustrates an exemplary design of the robotic arm 114, which may be part of the automated rehabilitation system 100. In one embodiment, the robotic arm may have at least one link, at least one effector, and at least one joint.

In some embodiments, the robotic arm may include a proximal link 131, an intermediate link 132, and an end effector 133. The proximal link may have a proximal joint 134 with at least one degree of freedom (e.g., 1, 2, 3, 4, 5, 6, or more degrees of freedom). The proximal link and the intermediate link may be connected by an intermediate joint 135 that has at least one degree of freedom (e.g., 1, 2, 3, 4, 5, 6, or more degrees of freedom). The intermediate link and the end effector may be connected by a distal joint 136 that has at least one degree of freedom (e.g., 1, 2, 3, 4, 5, 6, or more degrees of freedom).

In some embodiments, the end effector 133 may include at least one joint(s), each with at least one degree of movement. The end effector, in some embodiments, is capable of actively holding a part of the human body. The end effector can comprise a gripper. The gripper can comprise finger like projections that can be used to manipulate and hold a part of the human body. In some embodiments, a user is able to user various types of grippers for the purpose of different therapies. For example, a 2 finger gripper, 3 finger gripper, 4 finger gripper, and 5 finger gripper can be used. In some embodiments, the system comprises a plurality of different grippers that can be used depending on the type of therapy to be performed. For example, various configurations of finger grippers and non-finger grippers (e.g., vacuum cup) can be used. Other types of grippers are also contemplated (e.g., those not using finger style grippers).

In some embodiments, one or more markers may be placed on one or more robotic arm(s) 114 to assist the system 100 in determining the position of the robotic arm(s). In some embodiments, the system 100 is also capable of determining the position of the robotic arm(s) without the use of the marker(s).

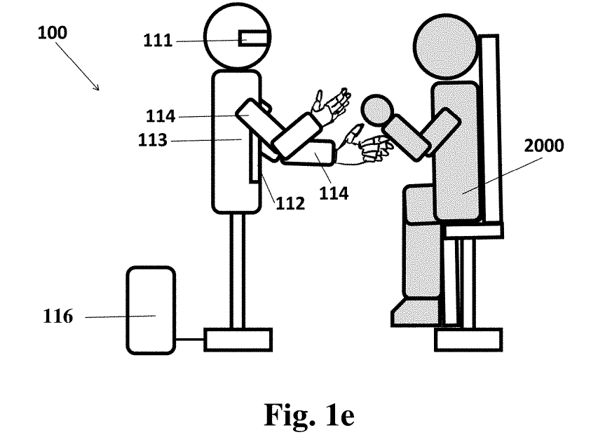

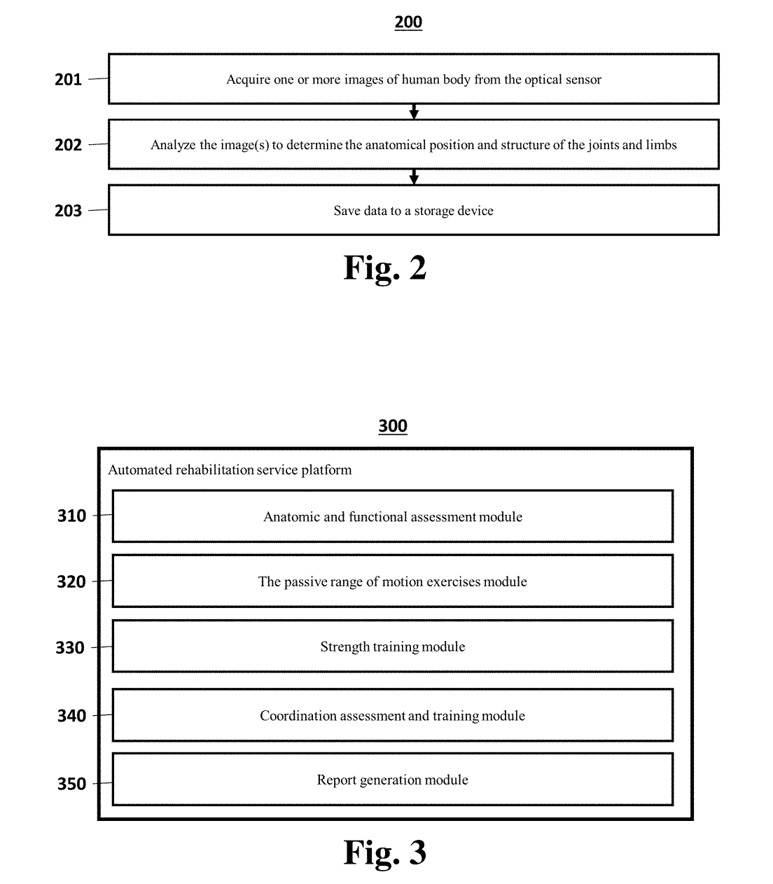

FIG. 2 is a flowchart of a method for determination of the anatomical structure 200 in accordance with one exemplary embodiment. In some embodiments, part of the method 200 can be executed using the automated rehabilitation system 100. The method can determine the anatomical structure of a part of the human body or entire human body.

The method 200 begins at block 201, where the system acquires one or more images of a human body from an optical sensor 111. The image(s) of the human body can be whole body image(s), or image(s) that shows only a part of the human body.

At block 202, the system 100 analyzes the acquired image(s) to determine the anatomical position and structure of the joints and limbs. In some embodiments, the system can determine the position of a patient's joints by analyzing body image of a patient, and then determine points in the image that could correspond to human joints. The anatomical structural data of the limbs can be derived from the joint position by drawing anatomically appropriate lines between joints.

In some embodiments, the system uses machine learning to determine the anatomical structure of a part of the human body. In some embodiments of a machine learning algorithm, the algorithm can determine a skeleton and joint points in a patient based on prior analysis of body images marked with joint positions. The machine learning can include providing the system with a large number of images of the human body with the joint positions and/or skeleton positions labeled. In other embodiments, the determination of the anatomical structure of human body can be performed based on other algorithms.

In some embodiments, determination of exact ranges between the joints can be determined by taking into account depth map data acquired by an optical sensor with a depth sensor.

In some embodiments, detachable marker(s) placed on the patient may aid the system in better determining the anatomical structure. In some embodiments, the detachable markers can transmit anatomic position data to the system either via a wired connection or wirelessly. In some embodiments, the detachable markers can emit optical signals to aid the system in better locating the anatomic structures using the system's optical sensor. In some embodiments, the system is also able to determine the anatomical structure of the human body based on the input from the optical sensor, without the aid of the detachable markers.

At block 203, the system saves the data obtained from to memory (e.g., a storage device).

In some embodiments, the method can assess the position of one or more joints, including the shoulder joint(s), the elbow joint(s), the wrist joint(s), the joint(s) of hand(s), the hip joint(s), the knee joint(s), the ankle joint(s), the pedal joint(s), joint(s) of the spine, joint(s) of the neck, and/or other joints of a patient.

In some embodiments, the method can assess the position of one or more limbs, including the right upper limb, right lower limb, left upper limb, and/or left lower limb of a patient.

FIG. 3 illustrates one embodiment of the automated rehabilitation service platform 300. In some embodiments, the control system 116 of the automated rehabilitation system 100 can be configured to operate part of the service platform.

In some embodiments, the platform 300 includes an anatomic and functional assessment module 310, the passive range of motion exercises module 320, strength training module 330, coordination training module 340, and report generation module 350.

The report generation module 350 can access the data stored in the system and generate reports containing the anatomic data, functional data, exercise data, and training data of the patients.

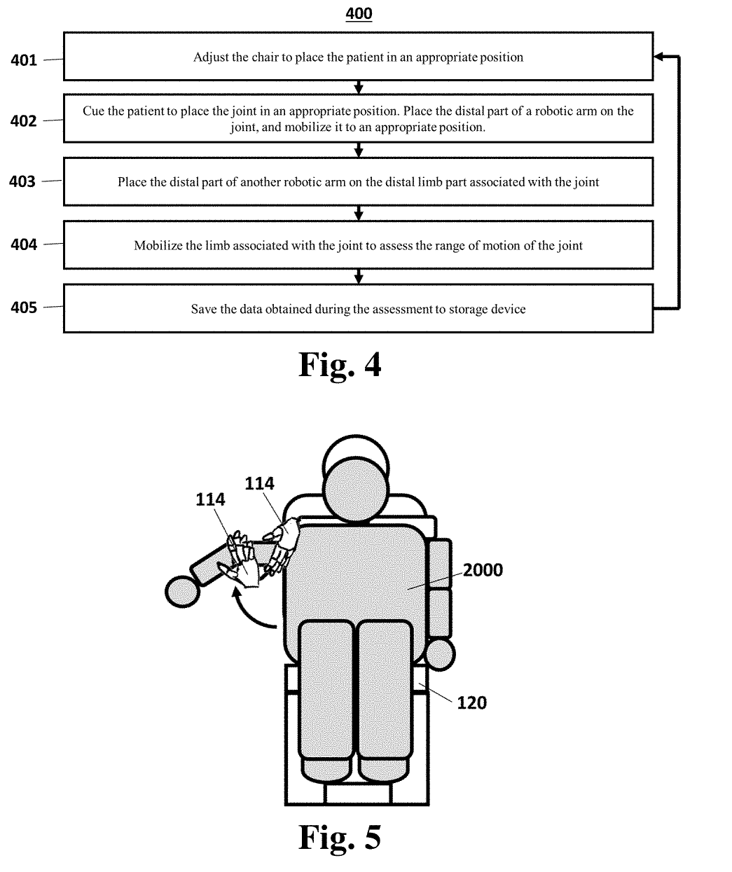

FIG. 4 is a flowchart of a method for determining the range of motion 400 in accordance with one exemplary embodiment. In some embodiments, the method can be executed using the automated rehabilitation system 100, and can be implemented as part of the anatomic and functional assessment module 310.

In some embodiments, the method can assess the range of motion of one or more joints, including the shoulder joint(s), the elbow joint(s), the wrist joint(s), the joint(s) of hand(s), the hip joint(s), the knee joint(s), the ankle joint(s), the pedal joints(s), joint(s) of the spine, joint(s) of the neck, and/or other joints of a patient.

The motion(s) that can be assessed for each joint can include: extension, flexion, abduction, adduction, rotation, and/or other movements of a joint.

For assessment of each joint, the method 400 begins at block 401, where a system adjusts a mobile chair 120 to put the patient in an appropriate position. In some embodiments, the system can rotate, recline, mobilize, and/or perform height adjustment to put the patient in an appropriate position.

To determine the position of the joints and limbs, in some embodiments, the system can utilize the method for determination of anatomical structure 200. The system, in some embodiments, could also determine the position of the joints and limbs using other methods.

At block 402, in some embodiments, the system cues the patient to place its joint in an appropriate position via a speaker 112 and/or a display unit 113 if the joint is not in an appropriate position. The system can place the distal part of a robotic arm on the joint when the joint is in an appropriate position. In some embodiments, the robotic arm 114 can attempt to move the joint to an appropriate position and then hold the joint in place.

At block 403, the system places the distal part of another robotic arm 114 on the distal limb part associated with the joint for assessment.

At block 404, the system can operate its robotic arms 114 to mobilize the limb associated with the joint to assess the range of motion of the joint. In some embodiments, the robotic arm 115 can move to mobilize the limb. The robotics arm can extend, flex, or rotate the limb during the assessment. The sensors on the system can record the input generated during the mobilization process. In some embodiments, the mobilization operation can be stopped if one of the following conditions is met: (1) Maximum range of motion has been reached as detected by the optical sensor and/or detachable markers, (2) Force sensor(s) on one or more robotic arms detects resistance meeting specified threshold, (3) The patient indicates pain, or (4) Other system input stopping mobilization. A maximum range of motion can be detected by a force sensor detecting a resistance level higher than a pre-set threshold when manipulating a limb. A resistance level higher than a pre-set threshold typically indicates that the limb is already at the end of its range of motion or there is limb spasticity or rigidity preventing further manipulation of the limb. In either situation, it can be dangerous to exert force to overcome the resistance. The threshold level can be adjusted by a user. A higher threshold setting would provide a more aggressive therapy, while a lower threshold setting may provide a safer therapy. In some embodiments, a more aggressive therapy can be more efficacious, but carries the risk of injury, while a safer therapy can be less efficacious but carries less risk of injury. The range of motion can be recorded as a distance the limb can move, range of angles through which the limb can move, and a degree of rotation through which the limb can be moved. The system can also record a resistance force detected by the force sensor throughout the range of motion and/or at the end of the range of motion, which can provide a clear condition of the joint.

At block 405, the system saves the data obtained from the sensors during the assessment process into memory (e.g., a storage device). The system can repeat the method 430 to assess the range of motion of other movement(s) of the joint, or to assess the range of motions of other joint(s) of the patients as specified in the system instruction.

FIG. 5 depicts one example of determining the range of motion of the right shoulder joint abduction of a patient 2000. In the example, the mobile chair system 120 would perform rotation, reclining, and/or height adjustment to put patient in a position appropriate for assessment of the range of motion of right shoulder joint abduction.

The system places the distal part of its left robotic arm 114 on top of the patient's right shoulder joint and attempts to hold it in place. The system then places the distal part of its right robotic arm 114 on the distal part of the patient's right forearm. The right robotic arm 114 then attempts to abduct the patient's right forearm to assess the range of motion for the right shoulder joint abduction.

In the example, the abduction of the right forearm by the robotic arm stops when the system detects one of the conditions as specified in block 404 of method 400. The system then records the range of motion achieved during the assessment into a storage device.

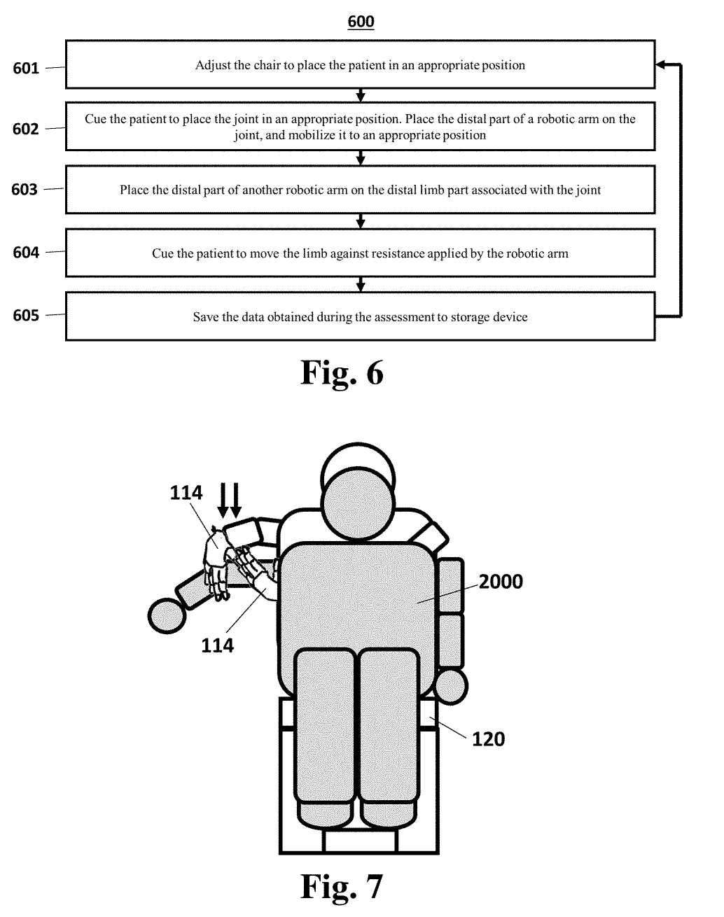

FIG. 6 is a flowchart of a method for functional assessment 600 in accordance with an exemplary embodiment. In some embodiments, part of the method 600 can be executed using the automated rehabilitation system 100, and can be implemented as part of the anatomic and functional assessment module 210.

In some embodiments, the method can assess the muscle strength associated with the movements of one or more joints, including the shoulder joint(s), the elbow joint(s), the wrist joint(s), the joint(s) of hand(s), the hip joint(s), the knee joint(s), the ankle joint(s), the pedal joints(s), joint(s) of the spine, joint(s) of the neck, and/or other joints of a patient.

In some embodiments, the motion(s) that can be assessed for each joint can include: extension, flexion, abduction, adduction, rotation, and/or other movements of a joint.

For assessment of each joint, in some embodiments, method 600 begins at block 601, where a system adjusts a mobile chair 120 to put the patient in an appropriate position. In some embodiments, the system can rotate, recline, mobilize, and/or perform height adjustment to put the patient in an appropriate position.

To determine the position of the joints and limbs, in some embodiments, the system can utilize the method for determination of anatomical structure 200. The system, in some embodiments, could also determine the position of the joints and limbs using other methods.

At block 602, in some embodiments, the system can cue the patient to place its joint in an appropriate position via a speaker 112 and/or a display unit 113 if the joint is not in an appropriate position. The system can place the distal part of a robotic arm on the joint when the joint is in an appropriate position. In some embodiments, the robotic arm 114 can attempt to move the joint to an appropriate position and then hold the joint in place.

At block 603, in some embodiments, the system places the distal part of another robotic arm 114 on the distal limb part associated with the joint for assessment.

At block 604, the system can operate its robotics arms 114 to assess the muscle strength of the limb associated with the joint. In some embodiments, the system cues the patient to move their limbs via a speaker 112 and/or a display unit 113. Then, the robotic arm 114 can apply appropriate resistance force, and record the force generated by the patient's assessed limb. In some embodiments, the assessment could stop if one of the following conditions is met: (1) certain period of time have elapsed, (2) the patient indicated that he/she have exerted his maximal muscle strength for the movement, or (3) other system input stopping assessment.

At block 605, the system saves the data obtained from the sensors during the assessment process into a storage device. The system can repeat the method 600 to assess the strength of other movements of the joint, or to assess the strength of other joint(s) of the patients as specified in the system instruction.

FIG. 7. depicts one example of determining the strength of the right shoulder joint abduction of a patient. In some embodiments, the mobile chair system 120 would perform rotation, reclination, and/or height adjustment to achieve a position appropriate for strength training of abduction of right shoulder joint.

In the example, the system places the distal part of its left robotic arm 114 below the patient's right shoulder joint and attempts to hold it in place. The system then places the distal part of its right robotic arm 114 on top of the distal part of the patient's right forearm.

The system can cue the patient to abduct its right shoulder joint via the speaker 112 and/or the display unit 113.

The right robotic arm 114 would then attempts to resist the patient's right shoulder abduction to assess the strength for the right shoulder joint abduction.

In the example, the assessment by the robotic arm would stop when the system detects conditions as specified in block 604 of method 600. The system would then record the patient's muscle strength during the assessment into a storage device.

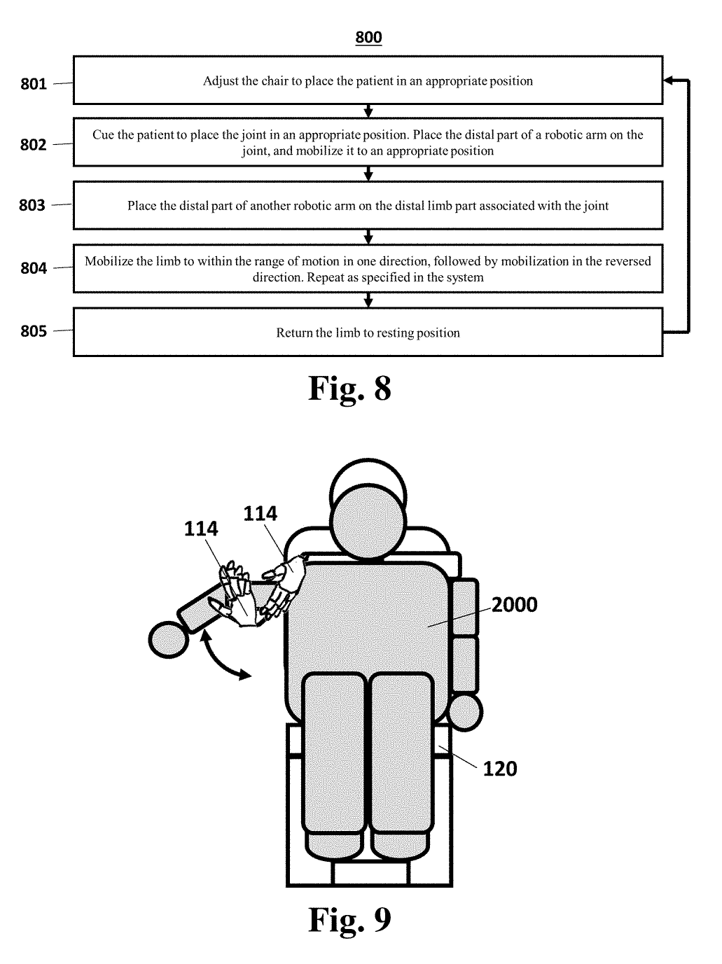

FIG. 8 is a flowchart of a method for performing the automated passive range of motion exercises (700) in accordance with some exemplary embodiments. In some embodiments, part of the method (800) can be executed using the automated rehabilitation system (100), and can be implemented as part of the passive range of motion exercises module (320).

In some embodiments, the method can perform the automated passive range of motion exercises for one or more joints, including the shoulder joint(s), the elbow joint(s), the wrist joint(s), the joint(s) of hand(s), the hip joint(s), the knee joint(s), the ankle joint(s), the pedal joints(s), joint(s) of the spine, joint(s) of the neck, and/or other joints of a patient.

The motion(s) that can be involved in the exercises of each joint can include: extension, flexion, abduction, adduction, rotation and/or other movements of a joint.

The system may utilize the limbs and joints anatomic and functional data stored in a storage device to adjust the process of the passive range of motion exercises.

For the automated passive range of motion exercises for each joint, method 800 begins at block 801, where a system adjusts a mobile chair 120 to put the patient in an appropriate position. In one embodiment, the system can rotate, recline, mobilize, and/or perform height adjustment to put the patient in an appropriate position.

To determine the position of the joints and limbs, in one embodiment, the system can utilize the method for determination of anatomical structure 200. The system, in some embodiments, can also determine the position of the joints and limbs using other methods.

At block 802, in some embodiments, the system cues the patient to place its joint in an appropriate position via a speaker 112 and/or a display unit 113 if the joint is not in an appropriate position. The system can place the distal part of a robotic arm on the joint when the joint is in an appropriate position. In some embodiments, the robotic arm 114 can attempt to move the joint to an appropriate position and then hold the joint in place.

At block 803, the system places the distal part of another robotic arm 114 on the distal limb part associated with the joint for exercises.

At block 804, the system operates its robotics arms 114 to perform the automated passive range of motion exercises for the joint. One robotic arm can mobilize the limb within its range of motion in one direction, followed by mobilization in the reversed direction. The robotic arms can repeat the mobilization as specified in the system for the purpose of the exercise.

At block 805, the system returns the limb to resting position. The system can repeat the method 700 to perform the automated passive range of motion exercises of other movements of the joint, or to perform the exercises for other joint(s) of the patients as specified in the system instruction.

FIG. 9 depicts one example of performing the automated passive range of motion exercises for the right shoulder joint for the abduction-adduction movement. In some embodiments, the mobile chair system 120 performs rotation, reclination, and/or height adjustment to put a patient 2000 in a position appropriate for the right shoulder joint passive range of motion exercises.

The system places the distal part of its left robotic arm 114 on the patient's right shoulder joint and attempts to hold it in place. The system then places the distal part of its right robotic arm 114 on the distal part of the patient's right forearm.

The robotic arm can then abduct the right forearm within the range of motion in one direction, followed by adduction of the right forearm. The system can repeat the abduction-adduction movement as specified in the system.

The passive range of motion exercises by the robotic arm can stop when the exercise process is completed, or when the exercise is stopped by instruction. The system would then return the right forearm to its original position.

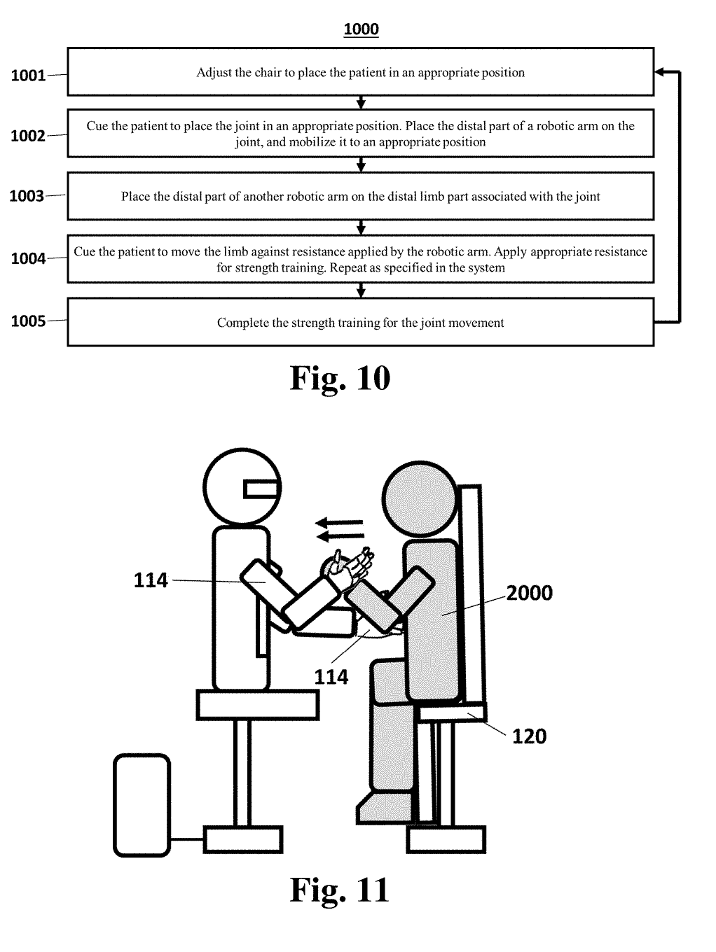

FIG. 10 is a flowchart of a method for performing automated strength training 1000 in accordance with an exemplary embodiment. In some embodiments, part of the method 1000 can be executed using the automated rehabilitation system 100, and can be implemented as part of the passive range of motion exercises module 330.

In some embodiments, the method can perform automated strength training for one or more joints, including the shoulder joint(s), the elbow joint(s), the wrist joint(s), the joint(s) of hand(s), the hip joint(s), the knee joint(s), the ankle joint(s), the pedal joints(s), joint(s) of the spine, joint(s) of the neck, and/or other joints of a patient.

The motion(s) that can be involved in the training of each joint can include: extension, flexion, abduction, adduction, rotation, and/or other movements of a joint.

The system may utilize the limbs and joints anatomic and functional data stored in a storage device to adjust the process of the training.

For automated strength training for each joint, method 1000 begins at block 1001, where a system adjusts a mobile chair 120 to put the patient in an appropriate position. In one embodiment, the system could rotate, recline, mobilize, and/or perform height adjustment to put the patient in an appropriate position.

To determine the position of the joints and limbs, in one embodiment, the system can utilize the method for determination of anatomical structure 200. The system, in some embodiments, could also determine the position of the joints and limbs using other methods.

At block 1002, in one embodiment, the system can cue the patient to place its joint in an appropriate position via a speaker 112 and/or a display unit 113 if the joint is not in an appropriate position. The system can place the distal part of a robotic arm on the joint when the joint is in an appropriate position. In one embodiment, the robotic arm 114 can attempt to move the joint to an appropriate position and then hold the joint in place.

At block 1003, the system places the distal part of another robotic arm 114 on the distal limb part associated with the joint for exercise.

At block 1004, the system can operate its robotics arms 114 to perform strength training of the joint. In one embodiment, the system can cue the patient to move its limbs via a speaker 112 and/or a display unit 113. Then, the robotic arm 114 can apply an appropriate resistance force to train muscle strength of the limb. The robotic arms can repeat the cue and resistance application as specified in the system for the purpose of the training.

At block 1005, the system completes the training. The system can repeat the method 1000 to perform strength training for other movements of the joint, or to perform training for other joint(s) of the patients as specified in the system instruction.

FIG. 11. depicts one example of performing the automated strength training for the left elbow joint for the flexion movement. The mobile chair system 120 can perform rotation, reclination, and/or height adjustment to put the patient 2000 in a position appropriate for strength training of right shoulder joint.

The system can then cue the patient to place its elbow joint in an appropriate position via a speaker 112 and/or a display unit 113. The system places the distal part of its left robotic arm 114 on the patient's elbow joint and attempts to hold it in place. The system then places the distal part of its right robotic arm 114 on the distal part of the patient's left hand, and cue the patient to flex his/her elbow.

The system can repeat the cue and resistance application as specified in the system.

In the example, the strength training by the robotic arm stops when the exercise process is completed, or when the exercise is stopped by instruction.

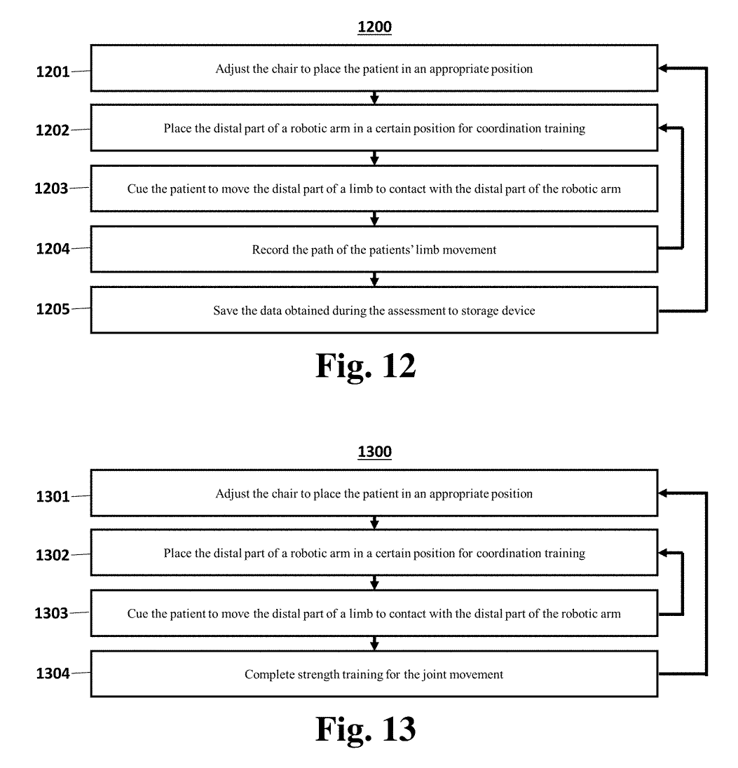

FIG. 12 is a flowchart of a method for coordination assessment 1200 in accordance with an exemplary embodiment. In some embodiments, the method can be executed using the automated rehabilitation system 100, and can be implemented as part of the coordination assessment and training module 340.

In some embodiments, the method can perform coordination assessment for one or more limbs, including the left upper limb, the right upper limb, left lower limb, and/or the right lower limb.

For assessment of each limb, method 1200 begins at block 1201, where the system adjusts a mobile chair 120 to put the patient in an appropriate position. In some embodiments, the system can rotate, recline, mobilize, and/or perform height adjustment to put the patient in an appropriate position.

To determine the position of the joints and limbs, in one embodiment, the system can utilize the method for determination of anatomical structure 200. The system can also determine the position of the joints and limbs using other methods.

At block 1202, the system places distal part of a robotic arm in a certain position for coordination training.

At block 1203, the system cues the patient to move the distal part of a limb to contact the distal part of a robotic arm. For example, the patient may move the limb in a certain path to contact the distal part of the robotic arm.

At block 1204, the system records the path of the patients' limb movement. In some embodiments, the path of the patient's limb movement can be determined by image(s) acquired by the optical sensor. In some embodiments, the path can also be determined based on joint position data detected by the detachable marker(s) placed on the patient.

The system can repeat the process from block 1202 to 1204 as specified in the system for the purpose of coordination assessment.

At block 1205, the system saves the data recorded to the internal storage. The system can repeat the method 1200 to perform coordination assessment for other limbs.

FIG. 13 is a flowchart of a method for coordination training 1300 in accordance with one exemplary embodiment. In some embodiments, part of the method 1300 can be executed using the automated rehabilitation system 100, and can be implemented as part of the coordination assessment and training module 340.

In some embodiments, the method can perform coordination training for one or more limbs, including the left upper limb, the right upper limb, left lower limb, and the right lower limb.

The system may utilize the limbs and joints anatomic and functional data stored in a storage device to adjust the process of the training.

For automated coordination training for each limb, method 1300 begins at block 1301, where a system adjusts a mobile chair 120 to put the patient in an appropriate position. In some embodiments, the system could rotate, recline, mobilize, and/or perform height adjustment to put the patient in an appropriate position.

To determine the position of the joints and limbs, in some embodiments, the system can utilize the method for determination of anatomical structure 200. The system, in some embodiments, can also determine the position of the joints and limbs using other methods.

At block 1302, the system places the distal part of a robotic arm in a certain position for coordination training.

At block 1303, the system cues the patient to move the distal part of a limb to contact with the distal part of a robotic arm. For example, the patient may move the limb in a certain path to achieve contact with the distal part of the robotic arm. In some embodiments, the system can record the data it obtains to an internal storage. Data can include the patient's conformance to the desired path and position of the limb.

The robotic arms can repeat the block 1302 and 1303 as specified in the system for the purpose of the coordination training.

At block 1304, the system completes the training. The system can repeat the method 1300 to perform coordination training for other limbs.

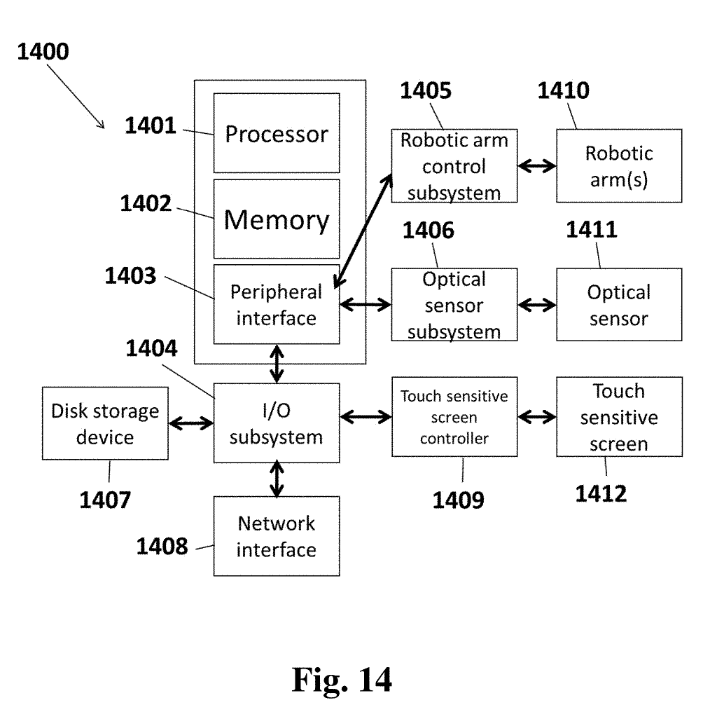

FIG. 14 depicts a computer system 1400 that, in some embodiments, can serve as the control system 116 for the automated rehabilitation system 100. The control system may be implemented in a computer system that includes one or more processors 1401, memory 1402, and a peripheral interface 1403. The memory 1402 may be of any type of medium capable of storing information accessible by the processor, being coupled to the processor, and storing instructions executable by the processor. The peripheral interface 1403 may be connected to input/output (I/O) subsystem 1404, robotic arm control subsystem 1405, and optical sensor subsystem 1406. The I/O subsystem may be connected to a disk storage device 1407, a network interface 1408, a touch sensitive screen controller 1409, or other input/output devices. The above system is intended to represent a machine in the exemplary form of a computer system that, in some embodiments, is capable of causing the machine to perform one or more methods discussed herein. The robotic arm control subsystem 1405, in some embodiments, is able to send and/or receive data from the robotic arm 1410. The optical sensor subsystem 1406, in some embodiments, is able to send and/or receive data from the optical sensor 1411. The touch sensitive screen controller 1409, in some embodiments, is able to send and/or receive data from the touch screen 1412)

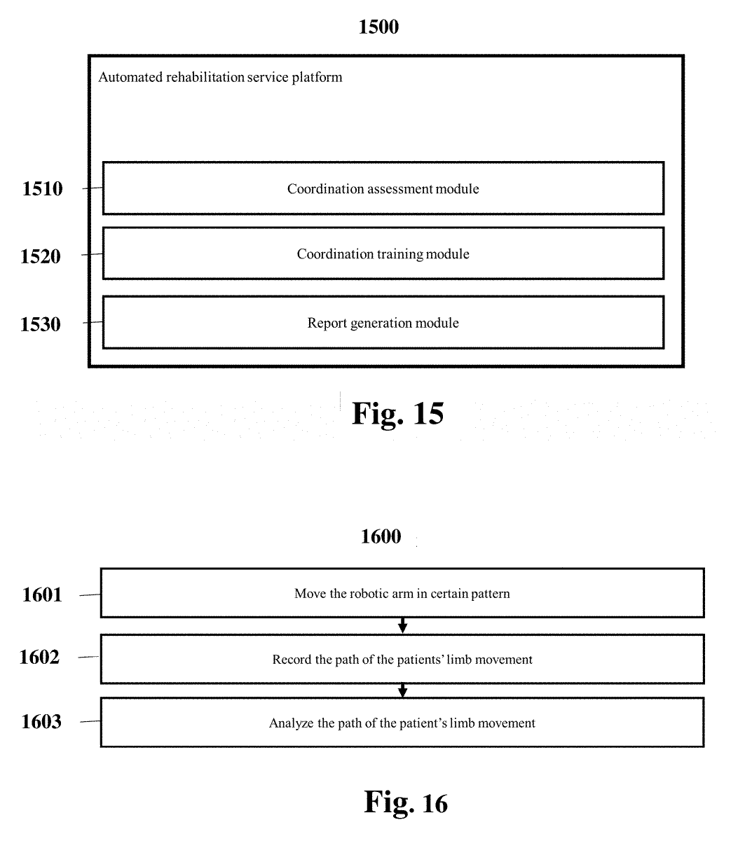

FIG. 15 illustrates an embodiment of an automated rehabilitation service platform (1500). In some embodiments, the control system 115 of the automated rehabilitation system 100 can be configured to operate part of the service platform.

In some embodiments, the platform 1500 includes a coordination assessment module 1510, coordination training module 1520, and report generation module 1530.

The report generation module 1530 can access the data stored in the system and generate reports containing the anatomic data, functional data, exercise data, and training data of the patients.

FIG. 16 is a flowchart of a method for coordination assessment 1600 in accordance with an exemplary embodiment. In some embodiments, the method can be executed using the automated rehabilitation system 100, and can be implemented as part of the coordination assessment module 1510 or the functional assessment module 310 of FIG. 3.

In some embodiments, the method can perform coordination assessment for one or more limbs, including the left upper limb, the right upper limb, left lower limb, and/or the right lower limb.

To determine the position of the joints and limbs, in some embodiments, the system can utilize the method for determination of anatomical structure 200. The system could also determine the position of the joints and limbs using other methods.

For assessment of each limb, method 1600 begins at block 1601, where the system moves the robotic arm in certain trajectory. In some embodiments, the patient may be cued to move the distal part of a limb to interact with the distal part of a robotic arm. For example, the patient may be cued to move the limb in a certain path following the movement of the distal part of the robotic arm.

At block 1602, the system records the path of the patients' limb movement. In one embodiment, the path of the patient's limb movement can be determined by image(s) acquired by the optical sensor. In some embodiments, the path can also be determined based on joint position data detected by the detachable marker(s) placed on the patient.

At block 1603, the system analyzes the path of the patients' limb movement. The system may determine the latency time, velocity, acceleration, accuracy, smoothness, and/or submovement of the patient's limb movement. In some embodiments, the system may compare the path of the patient's movement to the path supposed to be travelled by the patient to determine the patient's performance. In some embodiments, the system may also compare the path of the patient's movement to the path of the robotic arm's movement to determine the patient's performance. In some embodiments, the system may save the data recorded to the memory. The system can repeat the method 1600 to perform coordination assessment for other limbs.

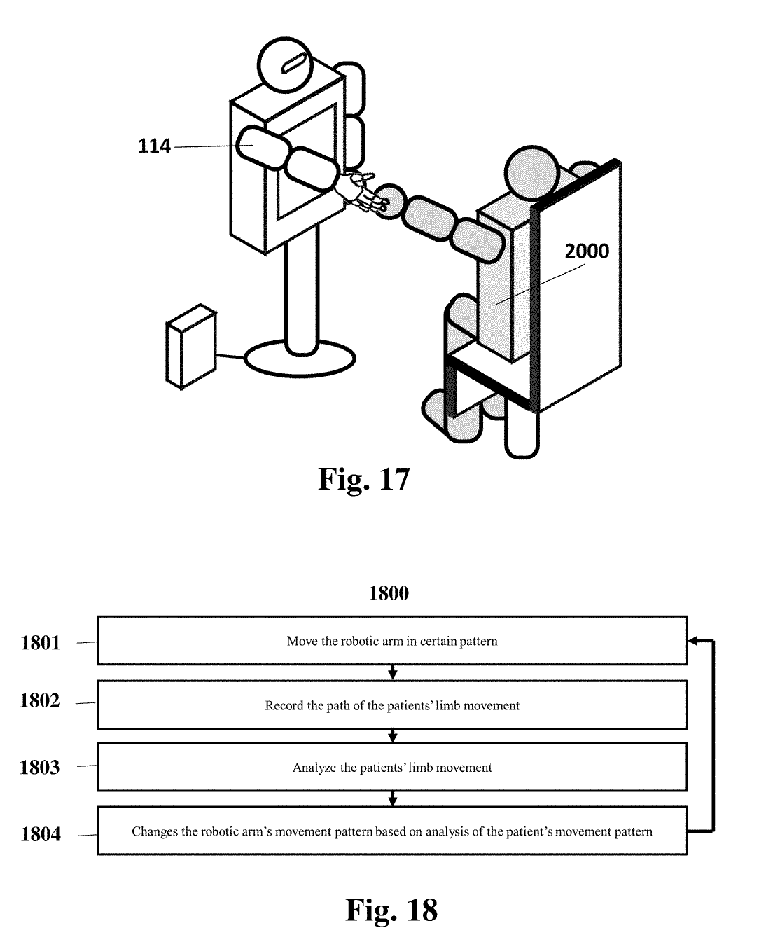

FIG. 17 depicts one example of coordination assessment of the left upper limb of a patient 2000. In the example, the system moves the distal part of its right robotic arm 114, and cues the patient to follow its right robotic arm with his/her left hand. The system then records the path of the patients' limb movement. The system would then analyze the path of patient's limb movement, and then store the results of the assessment into a memory. It will be appreciated that other therapies are also possible. For example, the system can cue the patient to follow the left robotic arm with the patient's right hand. For another example, the system can cue the patient to follow the right robotic arm with the patient's left foot.

FIG. 18 is a flowchart of a method for coordination training 1800 in accordance with one exemplary embodiment. In some embodiments, the method can perform coordination training for one or more limbs, including the left upper limb, the right upper limb, left lower limb, and the right lower limb. In some embodiments, the method can be executed using the automated rehabilitation system 100, and can be implemented as part of the coordination training module 1520 or the functional assessment module 210.

The system may utilize the limbs and joints anatomic and functional data stored in a storage device to adjust the process of the training.

For automated coordination training for each limb, the method 1800 begins at block 1801, where the system moves the robotic arm in a certain pattern. In some embodiments, the patient may be cued to move the distal part of a limb to interact with the distal part of a robotic arm, for purpose of coordination training. For example, the patient may be cued to move the limb in a certain path following the movement of the distal part of the robotic arm.

To determine the position of the joints and limbs, in one embodiment, the system can utilize the method for determination of anatomical structure 200. The system, in some embodiments, can also determine the position of the joints and limbs using other methods.

At block 1802, the system records the path of the patients' limb movement. In some embodiments, the path of the patient's limb movement can be determined by image(s) acquired by the optical sensor. In some embodiments, the path can also be determined based on joint position data detected by the detachable marker(s) placed on the patient.

At block 1803, the system analyzes the path of the patients' limb movement. The system may determine the latency time, velocity, acceleration, accuracy, smoothness, and/or submovement based on the patient's limb movement. In some embodiments, the system may compare the path of the patient's movement to the path supposed to be travelled by the patient to determine the patient's performance. In some embodiments, the system may also compare the path of the patient's movement to the path of the robotic arm's movement to determine the patient's performance. In some embodiments, the system may save the data recorded to the memory. The system can repeat the method 1500 to perform coordination assessment for other limbs.

At block 1804, the system changes the robotic arm's movement pattern based on analysis of the patient's movement pattern. In some embodiments, the system may increase the difficulty of the training if the patient is performing well during the training to present the patient with a greater challenge, thus increasing the efficacy of the training. Conversely, the system may decrease the difficulty of the training if the patient is performing poorly during the training. In some embodiments, the difficulty may be increased by increasing the velocity of the robotic arm's movement, increase the complexity of the robotic arm's movement pattern, and/or reducing the latency time of the robotic arm's movement. For example, the velocity can be increased; the latency time can be increased, the complexity of the movement pattern can be increased; the velocity and the complexity of the movement pattern can be increased; the velocity and the latency time can be increased; or the complexity and the latency time can be increased. In some embodiments, the difficulty may be reduced by decreasing the velocity of the robotic arm's movement, reducing the complexity of the robotic arm's movement pattern, and/or increasing the latency time of the robotic arm's movement. For example, the velocity can be decreased; the latency time can be decreased, the complexity of the movement pattern can be decreased; the velocity and the complexity of the movement pattern can be decreased; the velocity and the latency time can be decreased; or the complexity and the latency time can be decreased. The robotic arms can repeat the block 1801 to 1804 as specified in the system for the purpose of the coordination training.

The system can repeat the method 1800 to perform coordination training for other limbs.

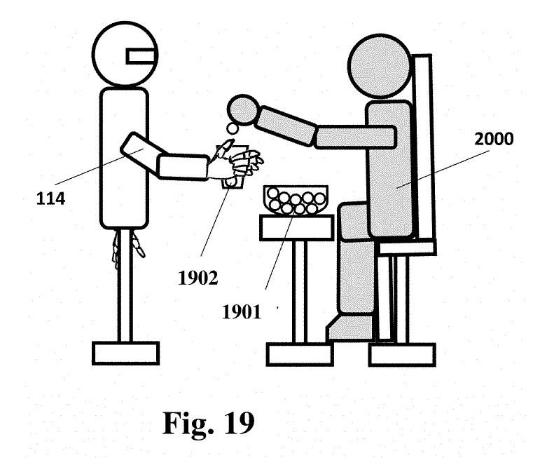

FIG. 19 depicts one example of coordination training of the left upper limb of a patient 2000. In the example, the system moves the distal part of its right robotic arm 114, and cues the patient to move the metal pieces, one at a time, from a bowl 1901 to a cup 1902 held by the right robotic arm. The system then records the path of the patients' limb movement. The system can then analyze the path of the patient's limb movement, and then store the results of the assessment into a memory. Based on the patient's performance, the system may change the velocity of the robotic arm's movement, complexity of the robotic arm's movement pattern, and/or the latency time of the robotic arm's movement, to present a greater/lesser challenge.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method of performing automated rehabilitation, the method comprising:
receiving data from one or more optical sensor;
determining a position of a part of the human body based on the data acquired by the optical sensor; and
controlling one or more robotic arms to mobilize a part of the human body based on the position of a part of the human body as determined by the processor.

2. The method of claim 1, wherein controlling one or more robotic arms comprises controlling the one or more robotic arms to perform a passive range of motion exercises by mobilizing a part of the human body in a reciprocal motion.

3. The method of claim 1, where controlling one or more robotic arms comprises controlling the one or more robotic arms to perform muscle strength training by applying resistance against exertion.

4. The method of claim 1, where controlling one or more robotic arms comprises controlling the one or more robotic arms and one or more optical sensor to perform coordination assessment by analyzing limb movement in a certain path while interacting with one or more robotic arms.

5. The method of claim 4, further comprising selecting a training routine based on the coordination assessment.

6. The method of claim 4, further comprising adjusting a training routine based on the coordination assessment.

7. The method of claim 1, where controlling one or more robotic arms comprises controlling the one or more robotic arms to perform coordination training by training a human to move limbs in a certain path by interacting with one or more robotic arms.

8. The method of claim 7, further comprising adjusting the coordination training based on a coordination assessment.

9. The method of claim 1, where controlling one or more robotic arms comprises controlling the one or more robotic arms to assess muscle strength by measuring the force of exertion against resistance.

10. The method of claim 9, further comprising selecting a training routine based on the coordination assessment.

11. The method of claim 9, further comprising adjusting a training routine based on the coordination assessment.

12. The method of claim 1, further comprising generating a rehabilitation report based on data obtained during rehabilitation.

13. The method of claim 1, further comprising determining a position of a part of the human body using machine learning.

14. The method of claim 1, further comprising adjusting therapy administered by the system.

15. The method of claim 1, further comprising determining latency time, velocity, acceleration, accuracy, smoothness, and/or submovement based on the patient's limb movement.

16. The method of claim 1, further comprising changing a velocity of the robotic arm's movement, complexity of the robotic arm's movement pattern, and/or the latency time of the robotic arm's movement based on analysis of limb movement.

17. A device for automated rehabilitation, comprising:
at least one robotic arm comprising a force sensor, at least one link, at least one effector, and at least one joint, with at least one degree of freedom for each joint;
an optical sensor;
a processor; and
memory coupled to the processor and storing instructions executable by the processor configured to cause the processor to:
receive data from the optical sensor;
determine a position of a part of the human body based on the data acquired by the optical sensor; and
control one or more robotic arms to mobilize a part of the human body based on a position of a part of the human body as determined by the processor.

18. The device of claim 17, further comprising instructions executable by the processor configured to cause the processor to control the one or more robotic arms to perform an automated passive range of motion exercises by mobilizing a part of the human body in a reciprocal motion.

19. The device of claim 17, further comprising instructions executable by the processor configured to cause the processor to select a passive range of motion exercise based on data acquired by the optical sensor.

20. The device of claim 17, further comprising instructions executable by the processor configured to cause the processor to adjust a passive range of motion exercise based on data acquired by the optical sensor.

21. The device of claim 17, further comprising additional instructions executable by the processor configured to cause the processor to perform muscle strength training by applying resistance against exertion.

22. The device of claim 17, further comprising additional instructions executable by the processor configured to cause the processor to control the one or more robotic arms to perform coordination assessment by analyzing limb movement in a certain path interacting with one or more robotic arms.

23. The device of claim 22, further comprising instructions executable by the processor configured to cause the processor to adjust an exercise based on the coordination assessment.

24. The device of claim 17, further comprising instructions executable by the processor to cause the processor to control one or more robotic arms to perform coordination training by training a user to move limbs in a certain path by interacting with one or more robotic arms.

25. The device of claim 17, further comprising instructions executable by the processor to cause the processor to adjust the coordination training based on data acquired by the optical sensor.

26. The device of claim 17, further comprising instructions executable by the processor to cause the processor to assess muscle strength by measuring the force of exertion against resistance.

27. The device of claim 17, further comprising instructions executable by the processor configured to cause the processor to generate a rehabilitation report from data received by the device.

28. The device of claim 17, further comprising instructions executable by the processor configured to cause the processor to determine a position of a part of the human body using machine learning.

29. The device of claim 17, which further comprising at least one height adjustable platform.

30. The device of claim 17, which further comprising at least one display unit.

31. The device of claim 17, which further comprising at least one speaker.

32. The device of claim 17, which further comprising at least one mobile chair.

33. The device of claim 32, in which the mobile chair is electronically controllable.

34. The device of claim 32, in which the mobile chair is height adjustable, rotatable and can be reclined.

35. The device of claim 17, which further comprising at least one detachable marker that can detect the position a part of the human body.

36. The device of claim 17, which further comprising at least one connection to a network.

37. The device of claim 17, further comprising instructions executable by the processor configured to cause the processor to determine latency time, velocity, acceleration, accuracy, smoothness, and/or submovement based on the patient's limb movement.

38. The device of claim 17, further comprising instructions executable by the processor configured to cause the processor to change a velocity of the robotic arm's movement, complexity of the robotic arm's movement pattern, and/or the latency time of the robotic arm's movement based on analysis of limb movement.

39. The device of claim 17, wherein the optical sensor comprises a depth sensor.
